(19) United States
(12) Patent Application Publication
(10) Pub. No.: US 2003/0036680 A1
(43) Pub. Date: Feb. 20, 2003

(54) Method and apparatus for thermal ablation of biological tissue using a scanning laser beam with real-time video monitoring and monitoring of therapeutic treatment parameters

(76) Inventor: Michael Black, Foster City, CA (US)

Correspondence Address: COOLEY GODWARD LLP, ATTN: PATENT GROUP, 11951 FREEDOM DRIVE, SUITE 1700, ONE FREEDOM SQUARE- RESTON TOWN CENTER, RESTON, VA, 20190-5061, US

(21) Appl. No.: 10/218,639

(22) Filed: Aug. 15, 2002

Related U.S. Application Data
(60) Provisional application No. 60/312,569, filed on Aug. 15, 2001.

(51) Int. Cl. A61B 1/04

(52) U.S. Cl. 600/108

(57) ABSTRACT

Method and apparatus for thermal ablation or coagulation of biological tissue using a scanning laser beam with real-time video monitoring and monitoring of therapeutic treatment parameters, such as temperature prior to or during treatment. In a preferred embodiment, a unique reflective optical delivery system is employed in conjunction with temperature control of the treatment area, and possibly, cryogenic treatment of the treatment area, to eliminate or reduce the need for anesthetics. All therapeutic parameters can be displayed on a video monitor, which is attached to a laser scanner. The reflective optics of the laser scanner can provide precise single-layer vaporization by the laser without thermal injury to the underlying tissue, and the video monitor allows a surgeon to monitor all therapeutic parameters both before and during a treatment procedure. The video monitor also can provide a three-dimensional view of the treatment area. This also can be videotaped for documentation purposes.

### PRIORITY

The present application claims priority from to commonly owned and assigned application Ser. No. 60/312,569, Attorney Docket No. RTEC-001/00US, entitled Method and Apparatus for Thermal Ablation of Biological Tissue with Scanning Laser Beam with Analyzing Parameters of Treatment Area, with Real-Time Video-Monitoring, which is incorporated herein by reference.

### FIELD OF THE INVENTION

This invention relates generally to the treatment of biological tissue using a laser device and, more particularly, to systems and methods that enable precise laser treatment of biological tissue surfaces with temperature control and analysis. In preferred embodiments, a video monitor may be attached to a laser scanner to provide stereo and three-dimensional images of an area of tissue under treatment. The monitor may also provide indications of the temperature of a target area to be treated.

### BACKGROUND OF THE INVENTION

Lasers have many useful applications for the treatment of tissue and other surfaces. For example, lasers have been used in the medical field to treat a wide variety of conditions including skin disorders, dental conditions, coronary conditions, vascular conditions, disorders of the reproductive tract, and vision impairment. In such applications, lasers have been used to destroy tissue through heat vaporization, to perform cold tissue ablation, and to provide for tissue coagulation.

In the area of skin disorders, however, it has been difficult to control many of the parameters relevant to laser therapy protocols, because it is often difficult to determine whether a treatment regimen is heating, burning, or affecting underlying or surrounding tissues. Thus, it is believed that those skilled in the art would find a laser beam delivery apparatus that provides physicians with increased control over a treatment setting and increased information about a therapeutic procedure to be quite useful.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

In one particularly innovative aspect, the present invention is directed to a laser beam delivery apparatus that provides a physician with a clear view of a target area to be treated and may also provide the physician with means for monitoring and controlling the temperature of the target area. These functionalities allow the physician to vaporize, for example, single tissue layers with reduced or eliminated thermal injury to surrounding tissues. These functionalities also may allow the physician to provide laser therapies for various conditions without the use of anesthetic.

In one presently preferred embodiment, a laser beam delivery apparatus in accordance with the present invention may comprise a coupling for receiving a beam carrier element, an optical viewing device, and a beam splitter in optical communication with the coupling and the optical viewing device. The beam splitter functions to direct a beam delivered by the beam carrier element to a target and to deliver light reflected from the target (i.e., an image of the target) to the optical viewing device. Those skilled in the art will appreciate that the optical viewing device may comprise a simple eyepiece and lens assembly, but it is presently preferred that the optical viewing device take the form of a CCD imager and an associated video monitor. The monitor may be mounted within a section of the housing of the beam delivery apparatus, or the monitor may comprise a separate unit. Those skilled in the art also will appreciate that the beam carrier element may comprise, for example, either an optical waveguide or fiber optic cable.

In another innovative aspect, a laser beam delivery apparatus in accordance with the present invention may further include a cryogenic fluid delivery system that comprises a portion of, or is carried by, the housing of the laser beam delivery apparatus. The cryogenic fluid delivery system enables a physician to controllably deliver a cryogenic liquid or gas to a target tissue area to control the temperature of the target tissue and surrounding tissues. In many situations, this may enable the physician to provide a desired laser therapy regimen without the use of anesthetic.

In still another innovative aspect, a laser beam delivery apparatus in accordance with the present invention may further include a temperature detector that is fixed within, or carried by, the housing of the device. The temperature detector may be coupled to a suitable microprocessor or central processing unit and may be used to provide or display an indication of tissue temperature at a target location on an associated video monitor that is carried by, or coupled to, the beam delivery apparatus.

In still another innovative aspect, a laser beam delivery apparatus in accordance with the present invention may comprise a scanning system for scanning a beam about the target. The scanning system may comprise, for example, a single mirror that is rotated or otherwise manipulated under microprocessor control, or the scanning system may comprise a plurality of mirrors that are manipulated under microprocessor control.

Accordingly, it is an object of the present invention to provide an improved laser beam delivery apparatus, or laser hand-piece, that may be used by physicians and others when conducting laser therapy procedures.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings wherein:

FIG. 1 is a schematic illustration of a laser beam delivery apparatus with a video camera and monitor in accordance with an embodiment of the present invention;

FIG. 2 is a schematic illustration of a laser beam delivery apparatus including a light source and cryogenic therapy device in accordance with another embodiment of the present invention;

FIG. 3 is a schematic illustration of a laser beam delivery apparatus including a reflector in accordance with another embodiment of the present invention;

FIG. 4 is a schematic illustration of a laser beam delivery apparatus including a temperature detector and microprocessing system in accordance with another embodiment of the present invention;

FIG. 5 is a schematic illustration of a laser beam delivery apparatus including an orthogonal scanner and a plurality of mirrors in accordance with another embodiment of the present invention;

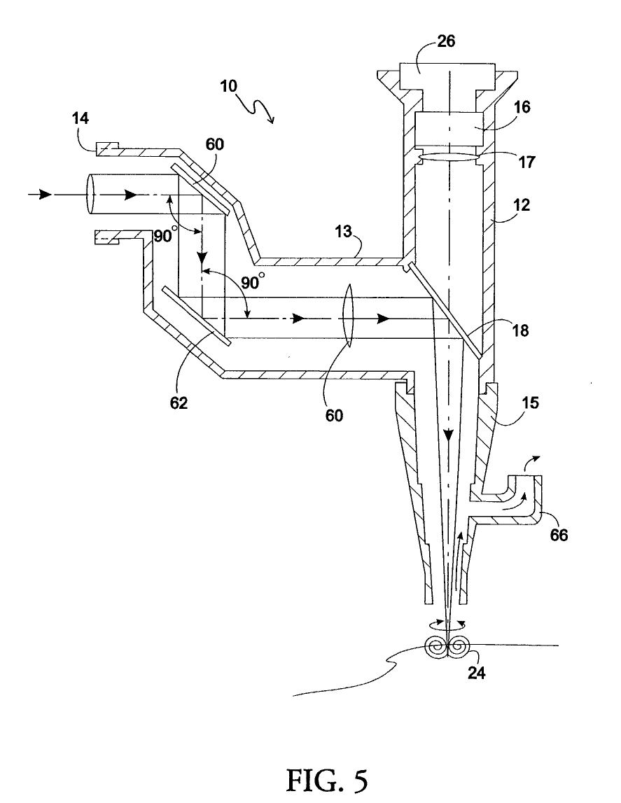

FIG. 6 is a schematic illustration of a laser beam delivery apparatus incorporating a cyclical scanner with a single mirror in accordance with another embodiment of the present invention; and

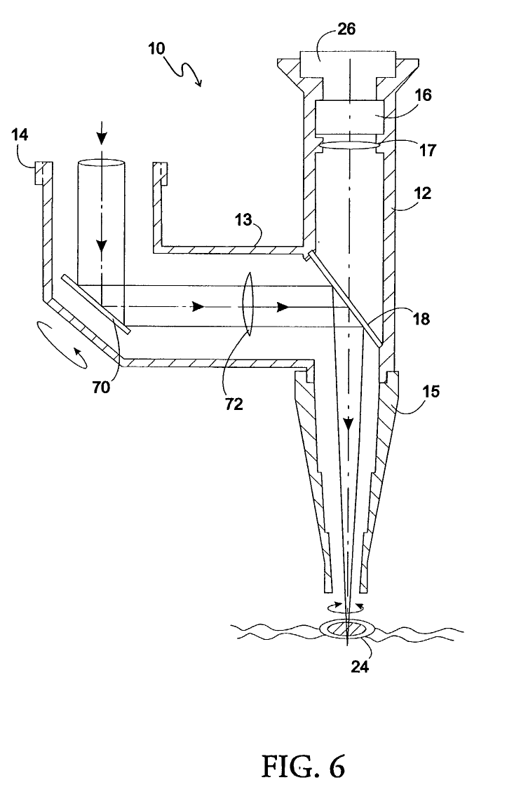

FIGS. 7(A), 7(B), 7(C), 7(D), 7(E), 7(F) AND 7(G) are graphic representations of various scanning modes that may be achieved using a laser beam delivery apparatus in accordance with selected embodiments of the present invention.

### DETAILED DESCRIPTION

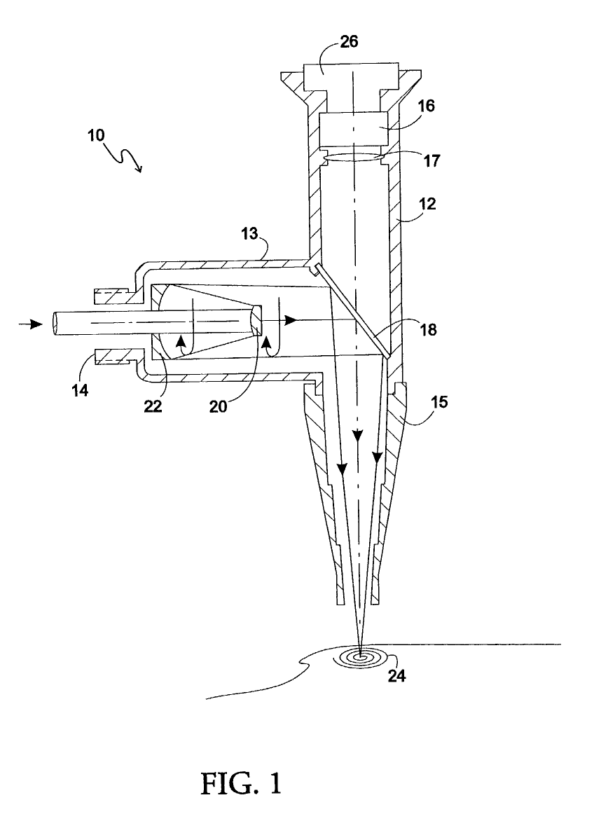

Referring now to the drawings, where like or similar elements are designated with identical reference numerals throughout the several views, and referring in particular to FIG. 1, a laser beam deliver apparatus 10 in accordance with a first embodiment of the present invention may comprise a housing 12 sized for manipulation by a human hand (not shown). The housing 12 may be formed as a unitary element, or the housing 12 may comprise a main body section 13 and distal sleeve section 15. The housing 12 preferably further includes a connector 14 for coupling to, or engaging, a beam carrier element, such as an optical waveguide or fiber optic cable (not shown), and the housing 12 may have mounted therein a CCD imager 16, an associated focusing lens 17, a beam splitter 18, and first and second mirrors 20 and 22. The beam splitter 18 functions to deliver a beam provided by the beam carrier element (not shown) to a target 24 and to deliver light reflected from the target 24 (i.e., an image of the target 24) to the CCD imager 16. The first mirror 20 may comprise a convex mirror, and the second mirror 22 may comprise a concave mirror such that the mirrors 20 and 22 function to focus the beam delivered by the beam carrier element (not shown) upon the target 24.

In a presently preferred embodiment, the CCD imager 16 may comprise a portion of a video-monitoring system, such as the EndoView system produced by Urohealth Surgical Division. That system includes an LCD monitor 26 that is coupled electronically to the CCD imager 16 and may be mounted within the housing 12 of the beam delivery apparatus 10. The beam splitter 18 may be purchased from Balzers Thin Films, Inc., of Golden, Colo. The treatment beam (not shown) delivered by the beam carrier element (not shown) can be a CO2 laser beam, or any other laser beam, including, for example, Argon, KTP, Nd:YAG, Erbium, etc. If the treatment beam is invisible, for example, if the treatment beam has a frequency falling within the infrared spectrum, then a guiding beam can be employed, and the guiding beam can be red, green, orange, yellow, blue or any other color available in the market.

Mirrors 20 and 22 preferably comprise a portion of a scanning system (not shown) and preferably can be manipulated or rotated, as described in U.S. Pat. No. 4,923,263, issued to Johnson, which is hereby incorporated by reference.

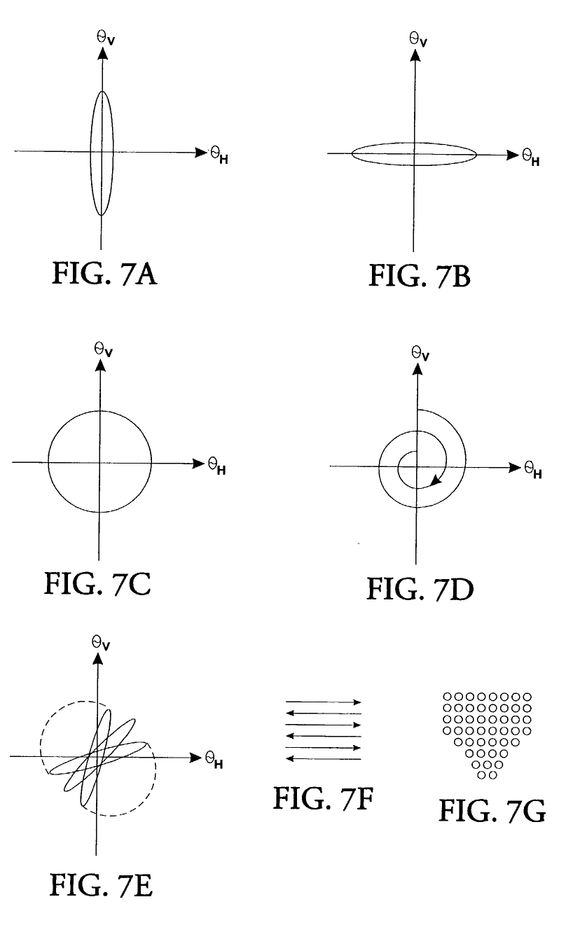

Those skilled in the art will appreciate that by changing the input parameters provided at a control unit (not shown) of the beam deliver apparatus 10, it is possible to create a variety of different treatment patterns, at the discretion of the laser operator or surgeon. Such patterns also can be pre-programmed prior to surgery and displayed at the operating site, and several exemplary scanning patterns are illustrated in FIGS. 7A-G. The scanning mechanism employed by this novel apparatus can contain two optical elements, such as those contained in the Accuscan laser scanner produced by Reliant Technologies, Foster City, Calif. That scanner can combine simultaneously a variety of different lasers for ablation (CO2, Erbium, or Holmium lasers) and coagulation (Nd:YAG, Argon, KTP) and at the same time can scan and focus such laser beams. The scanning mechanism also could be implemented using a SWIFTLASE or SILKTOUCH scanner produced by Sharplan Laser Industries, Allendale, N.J. Such systems, however, can be used with only one specific treatment laser beam that is selected by the operator or surgeon prior to surgery, because they utilize a focusing lens of specific transparent material for transmission of a specific beam.

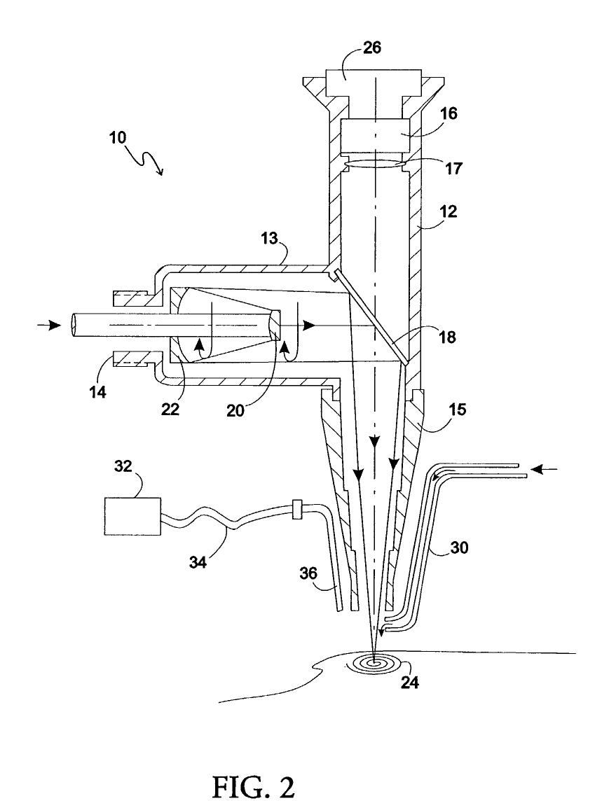

Turning now also to FIG. 2, in a presently preferred embodiment, the beam delivery apparatus 10 may further comprise a cryogenic fluid delivery apparatus 30 that is carried by, or formed within, the sleeve portion 15 of the housing 12. The cryogenic fluid delivery apparatus 30 preferably has a special configuration at the treatment site, which allows cooling gas to concentrate at a specific point or, alternatively, to concentrate within a variety of different areas having different shapes and sizes. Further, in a preferred form, the cryogenic fluid delivery apparatus 30 can be switched easily from one fluid delivery configuration to another.

As shown in FIG. 2, the beam delivery apparatus 10 also may include a light channel 36 for illuminating a target 24. The light channel 36 can be connected to a conventional light source 32, such as one produced by Wolf Inc., Rosemont, Ill., via a suitable fiberoptic cable 34. The configuration and use of light channels of the type described herein are well known in the art.

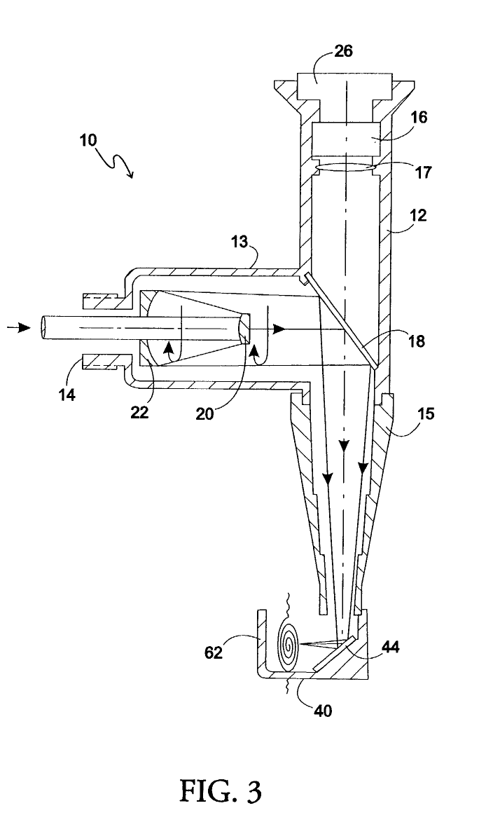

Turning now also to FIG. 3, the sleeve portion 15 of the housing 12 may further include a distal extension 40 with a holding hook or flange 42 that is used for ensuring proper positioning of an area of tissue to be treated. In embodiments, such as that shown in FIG. 3, the distal extension 40 may extend laterally from a center line (not shown) of the sleeve 15 and may have mounted therein a reflector or mirror 44 for directing the treatment beam toward the tissue to be treated.

The sleeve 15 may take the form of a standard otoscope cannula, and may be identical in design to those produced by Heine USA Ltd. When configured in this manner, the beam delivery apparatus 10 will allow physicians to treat numerous conditions including, for example, otitis media in children and adults. In such embodiments, the distal portion of the sleeve 15 can be used not only to protect surrounding tissues from thermal damage, but also to guide the treatment beam to a desired area.

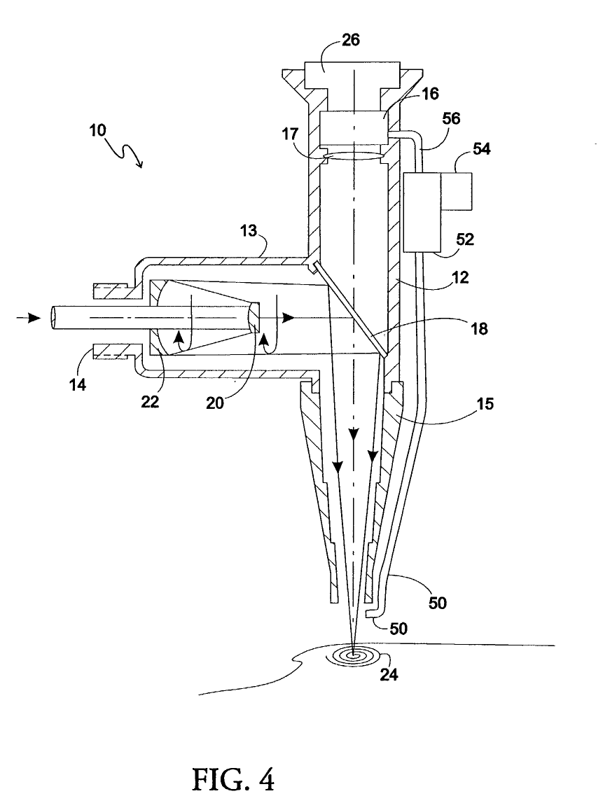

Turning now also to FIG. 4, a laser beam delivery apparatus 10 in accordance with the present invention may further include a thermodetector 50 that is coupled to the video monitor 26 via a microprocessor 52. The thermodetector 50 is available, for example, from Exergen Corporation, Newton, Mass., and is preferably located on a front end of the sleeve 15 of the beam delivery apparatus 10. The thermodetector 50 may be configured for physical contact with biological tissue at or near the target area 24, or the thermodetector 50 can be configured for indirect, non-contact monitoring of the tissue at or near the target 24.

Use of the thermodetector 50 and related circuitry allows for indications of tissue temperatures at the target 24 to be displayed on the video monitor 26. This enables real-time verification of tissue temperatures and conditions during treatment regimens, and when used in conjunction with a cryogenic fluid delivery system 30 (described with reference to FIG. 2), will enable physicians to control tissue temperatures during a procedure to prevent or reduce overheating of, and thermal damage to, surrounding and underlying treatment surfaces. This also may allow physicians to forgo the use of anesthetics when performing numerous procedures.

This temperature control capability may be very important, because an apparatus 10 in accordance with the present invention may, as described above, include a cryogenic fluid delivery system 30 that comprises a plurality of angular elements (not shown) to provide a variety of patterns for cryogenic treatment of biological tissues. Use of the cryogenic fluid delivery system 30 may allow physicians to perform procedures without the use of anesthetic, because in such procedures the physician can use a cooling gas to lower the temperature of a target area 24 before treatment, and the physician can monitor the temperature of the target area 24 during treatment to ensure that the target area temperature stays within a selected range that is tolerable to the patient.

Those skilled in the art will appreciate that, when using an apparatus 10 in accordance with various embodiments of the present invention, it is possible to view a target area 24 in either two or three dimensions. Moreover, those skilled in the art will appreciate that by modifying the monitor 26 and utilizing 3-D view eyeglasses, such as CrystalEyes, produced by StereoGraphics, San Rafael, Calif., or Virtual I-glasses produced by Virtual I-O, Inc., it is possible to provide a physician with both planar and three-dimensional views of a target area 24, and that under such conditions the physician should have increased control of the penetration depth used within a given procedure. This, of course, enables the physician to deliver a three-dimensional treatment regiment to a target location 24, if that is desired.

Accordingly it is a primary object of the present invention to provide a method and apparatus for treating biological tissue surfaces with lasers and real-time video monitoring. Moreover, laser systems in accordance with various embodiments of the present invention can provide a physician (or other device operator) with significant information during a treatment regimen. This information may include, for example, all relevant device parameters, such as laser type, the laser power or energy setting, total time of laser during treatment, the number of pulses provided to a target area within prescribed time limits and over the course of an entire procedure; the temperature of tissue within and surrounding a target area prior to and during treatment; the temperature tissue following cryogenic treatment; and the like. Thus, devices in accordance with various aspects of the present invention will provide physicians, and other relevant personnel, with improved information about, and significantly increased control over, a given therapy regimen.

Devices of the type described and claimed herein can be used to treat numerous conditions, including otitis media, which accounts in the U.S. for approximately 30,000,000 patient visits per year among children and adults.

It will be clear to one skilled in the art, that the above embodiments may be altered in many ways without departing from the scope of the invention. For example, many various laser scanning mechanisms can be used, many different video monitoring systems can be employed, many biological and non-biological surfaces can be treated, many different laser sources (continuous wave or pulse) can be used, and many different medical conditions can be treated. Accordingly, those skilled in the art will appreciate that the invention is not to be limited to the particular forms or methods disclosed herein, but rather, is to cover all modifications, equivalents, and alternatives falling within the spirit and scope of the appended claims.

What is claimed is:

1. A laser beam delivery apparatus comprising:
a housing sized for manipulation by a human hand, said housing having provided therein
a coupling for receiving a beam carrier element;
an optical viewing device; and
a beam splitter in optical communication with said coupling and said optical viewing device for directing a beam delivered by the beam carrier element to a target and for delivering an image of said target to said optical viewing device.

2. The laser beam delivery apparatus of claim 1, wherein the beam carrier element is selected from the group consisting of optical fibers and wave-guides.

3. The laser beam delivery apparatus of claim 1, wherein the optical viewing device comprises a CCD imaging element and a video monitor.

4. The laser beam delivery apparatus of claim 1, wherein the optical viewing device comprises an eyepiece including a lens assembly.

5. The laser beam delivery apparatus of claim 1 further comprising a first convex mirror and a second concave mirror, said mirrors being mounted within said housing and arranged to focus the beam delivered by said beam carrier element upon said target.

6. The laser beam delivery apparatus of claim 5, wherein said first convex mirror is configured for mechanical manipulation under microprocessor control to enable scanning of said beam about said target.

7. A laser beam delivery apparatus comprising:
a housing sized for manipulation by a human hand, said housing including a connector for receiving and engaging a beam carrier element;
a CCD imager mounted within said housing;
a video monitor coupled to said CCD imager;
a beam splitter mounted within said housing for delivering a beam provided by said beam carrier element to a target and for passing light reflected by said target to said CCD imager;
a system for focusing said beam provided by said beam carrier element upon said target; and
a cryogenic fluid delivery system carried by said housing for delivering a cold fluid to said target.

8. The laser beam delivery apparatus of claim 7 further comprising a light system for illuminating said target.

9. The laser beam delivery apparatus of claim 8, wherein said light system comprises a light channel that can be coupled to a fiber optic cable.

10. The laser beam delivery apparatus of claim 7, wherein said fluid is selected from the group consisting of cryogenic liquids and gases.

11. The laser beam delivery apparatus of claim 7, wherein said system for focusing said beam upon said target comprises a convex mirror and a concave mirror disposed within a path of said beam delivered by said beam carrier element.

12. The laser beam delivery apparatus of claim 7 further comprising a lens for focusing said light reflected by said target upon said CCD imager.

13. The laser beam delivery apparatus of claim 7, wherein said housing further comprises a distal flange for assisting in positioning said target.

14. The laser beam delivery apparatus of claim 13, wherein said distal flange extends laterally from a distal portion of said housing and has fixed therein a mirror for directing said beam to a target that is positioned laterally from a central axis of said housing.

15. The laser beam delivery apparatus of claim 7, wherein said video monitor is affixed within a proximal portion of said housing.

16. A laser beam delivery apparatus comprising:
a housing sized for manipulation by a human hand, said housing including a connector for receiving and engaging a beam carrier element;
a CCD imager mounted within said housing;
a video monitor coupled to said CCD imager;
a beam splitter mounted within said housing for delivering a beam provided by said beam carrier element to a target and for passing light reflected by said target to said CCD imager;
a system for focusing said beam provided by said beam carrier element upon said target; and
a temperature detector carried by said housing for generating signals indicative of a temperature of said target, said temperature detector being coupled via a processing element to said video monitor such that target temperature information may be displayed on said video monitor during a laser treatment procedure.

17. The laser beam delivery apparatus of claim 16 further comprising:
a light system carried by said housing for illuminating said target; and
a cryogenic fluid delivery system carried by said housing for delivering a cold fluid to said target.

18. The laser beam delivery apparatus of claim 17, wherein said fluid is selected from the group consisting of cryogenic liquids and gases.

19. The laser beam delivery apparatus of claim 16 further comprising means for scanning said beam in a pattern upon said target.

20. The laser beam delivery apparatus of claim 16, wherein said housing further comprises a smoke evacuation port.

21. A hand-held laser beam delivery apparatus comprising:
a housing having an optical viewing device and a connector for engaging a beam carrier element provided therein;
a beam splitter fixed within said housing along an optical path between an output of said beam carrier element and a target, said beam splitter being configured to direct a beam provided by said beam carrier element to said target and to deliver light reflected from said target to said optical viewing device; and
a beam scanning means provided along said optical path between said output of said beam carrier element and said beam splitter.

22. The hand-held laser beam delivery apparatus of claim 21, wherein said beam scanning means comprises a plurality of mirrors that are configured for spatial manipulation under microprocessor control and a focusing element.

23. The hand-held laser beam delivery apparatus of claim 21, wherein said beam scanning means comprises a single mirror configured for rotation under microprocessor control and a focusing element.

24. The hand-held laser beam delivery apparatus of claim 21 further comprising a temperature detector carried by said housing for generating signals indicative of a temperature of said target, said temperature detector being coupled via a processing element to a video monitor comprising a portion of said optical viewing device such that target temperature information may be displayed on said video monitor during a laser treatment procedure.

25. The hand-held laser beam delivery apparatus of claim 21 further comprising:
a light system carried by said housing for illuminating said target; and
a cryogenic fluid delivery system carried by said housing for delivering a cold fluid to said target.
